(19) United States
(12) Patent Application Publication
(10) Pub. No.: US 2026/0224142 A1
(43) Pub. Date: Aug. 6, 2026

(54) MENTAL STATE ESTIMATION DEVICE, MENTAL STATE ESTIMATION METHOD, AND STORAGE MEDIUM

(71) Applicant: NEC Corporation, Tokyo (JP)

(72) Inventor: Masanori TSUJIKAWA, Tokyo (JP)

(73) Assignee: NEC Corporation, Tokyo (JP)

(21) Appl. No.: 19/147,742

(22) PCT Filed: Jan. 19, 2023
(86) PCT No.: PCT/JP2023/001501

§ 371 Date: Jul. 14, 2025

(51) Int. Cl. A61B 5/16; A61B 5/00; A61B 5/11

(52) U.S. Cl. CPC A61B 5/165; A61B 5/0077; A61B 5/1128; A61B 5/7267; A61B 5/742

(57) ABSTRACT

A mental state estimation device 1Y mainly include a mental state feature amount acquisition means 21Y, a face direction feature amount acquisition means 23Y, and a mental state estimation means 22Y. The mental state feature amount acquisition means 21Y is configured to acquire a mental state feature amount as a feature amount calculated from a face image of a subject and used for estimation of a mental state of the subject. The face direction feature amount acquisition means is configured to acquire a face direction feature amount as a feature amount related to a face direction of the subject and calculated from the face image. The mental state estimation means 22Y is configured to estimate the mental state based on the mental state feature amount and the face direction feature amount. The mental state estimation device enables support for decision-making based on internal mental states.

### TECHNICAL FIELD

The present disclosure relates to a technical field of a mental state estimation device, a mental state estimation method, and a storage medium that perform processing related to estimation of a mental state.

### BACKGROUND

A device or a system that estimates a mental state of a subject based on a face image obtained by imaging the subject is known. For example, Patent Literature 1 discloses a device that estimates a subject's drowsiness from a face image (face video) of the subject imaged by a camera. Non-Patent Literature 1 discloses a technology for generating, from a face image, another image in which a direction of a face is different.

### CITATION LIST

### Patent Literature

### Patent Literature 1: WO 2019/123569 A1

### Non-Patent Literature

### Non-Patent Literature 1: Hang Zhou et al., Rotate-and-Render: Unsupervised Photorealistic Face Rotation From Single-View Images, CVPR2020.

### SUMMARY

### Problem to be Solved

In Patent Literature 1, the drowsiness can be estimated with high accuracy even in a face video of a low frame rate by capturing, as a feature of the drowsiness, movement of an eyelid slower than blinking. On the other hand, in a case where appearance of eyes is different between a time of training of a model and a time of estimation using the model due to an installation position of the camera, or the like, there is a possibility that the estimation accuracy is deteriorated.

In view of the problem described above, one object of the present disclosure is to provide a mental state estimation device, a mental state estimation method, and a storage medium capable of estimating a mental state of a subject with high accuracy.

### MEANS FOR SOLVING THE PROBLEM

In one mode of the mental state estimation device, there is provided a mental state estimation including:
a mental state feature amount acquisition means for acquiring a mental state feature amount as a feature amount calculated from a face image of a subject and used for estimation of a mental state of the subject;
a mental state estimation means for acquiring a predetermined number of estimation results of the mental state based on the mental state feature amount and a predetermined number of mental state estimation models each trained using face images in which face directions are different; and
an integration means for generating an integrated estimation result obtained by integrating the predetermined number of the estimation results of the mental state.

In another mode of the mental state estimation device, there is provided a mental state estimation including:
a mental state feature amount acquisition means for acquiring a mental state feature amount as a feature amount calculated from a face image of a subject and used for estimation of a mental state of the subject;
a face direction feature amount acquisition means for acquiring a face direction feature amount as a feature amount related to a face direction of the subject and calculated from the face image; and
a mental state estimation means for estimating the mental state based on the mental state feature amount and the face direction feature amount.

In one mode of the mental state estimation method by a computer, the mental state estimation method includes:
acquiring a mental state feature amount as a feature amount calculated from a face image of a subject and used for estimation of a mental state of the subject;
acquiring a predetermined number of estimation results of the mental state based on the mental state feature amount and a predetermined number of mental state estimation models each trained using face images in which face directions are different; and
generating an integrated estimation result obtained by integrating the predetermined number of the estimation results of the mental state.

In another mode of the mental state estimation method by a computer, the mental state estimation method includes:
acquiring a mental state feature amount as a feature amount calculated from a face image of a subject and used for estimating a mental state of the subject;
acquiring a face direction feature amount as a feature amount related to a face direction of the subject and calculated from the face image; and
estimates the mental state based on the mental state feature amount and the face direction feature amount.

It is noted that the “computer” includes any electronic device (may be a processor included in the electronic device) and may be configured by a plurality of electronic devices.

In one mode of the storage medium, there is provided a storage medium storing a program for causing a computer to execute processing including:
acquiring a mental state feature amount as a feature amount calculated from a face image of a subject and used for estimation of a mental state of the subject;
acquiring a predetermined number of estimation results of the mental state based on the mental state feature amount and a predetermined number of mental state estimation models each trained using face images in which face directions are different; and
generating an integrated estimation result obtained by integrating the predetermined number of the estimation results of the mental state.

In one mode of the storage medium, there is provided a storage medium storing a program for causing a computer to execute processing including:
acquiring a mental state feature amount as a feature amount calculated from a face image of a subject and used for estimating a mental state of the subject;
acquiring a face direction feature amount as a feature amount related to a face direction of the subject and calculated from the face image; and
estimating the mental state based on the mental state feature amount and the face direction

### EFFECT

An example advantage according to the present invention is to estimate the mental state of a subject with high accuracy.

### BRIEF DESCRIPTION OF THE DRAWINGS

FIG. 1 illustrates a schematic configuration of a mental state estimation system according to a first example embodiment.

FIG. 2 illustrates an example of a hardware configuration of a mental state estimation device common to each example embodiment.

FIG. 3A is a face image of a subject facing a front direction relative to a camera that performs imaging.

FIG. 3B is a face image of a subject facing upward at a predetermined angle relative to the camera that performs imaging.

FIG. 3C is a face image of a subject facing rightward at a predetermined angle relative to the camera that performs imaging.

FIG. 4 is an example of functional blocks of a mental state estimation device related to training of a mental state estimation model according to the first example embodiment.

FIG. 5A illustrates a distribution related to directions of a face in original face images stored in a training data storage unit.

FIG. 5B illustrates a distribution related to directions of the face in augmented face images generated from the original face images.

FIG. 6 is an example of a flowchart related to the training of the mental state estimation model executed by the mental state estimation device in the first example embodiment.

FIG. 7 is an example of functional blocks of the mental state estimation device related to estimation of a mental state in the first example embodiment.

FIG. 8 is an example of a flowchart executed by the mental state estimation device related to the estimation of the mental state in the first example embodiment.

FIG. 9A is a diagram in which a generation environment of an evaluation data set is observed from a direction in which a subject exists.

FIG. 9B is a diagram in which the generation environment of the evaluation data set is observed from a side of the subject.

FIG. 10A is a graph indicating evaluation results of a present disclosure method and a comparative method in a case where face images generated by a camera 5A are used as inputs to the mental state estimation model.

FIG. 10B is a graph indicating evaluation results of the present disclosure method and the comparative method in a case where face images generated by a camera 5B are used as inputs to the mental state estimation model.

FIG. 11A is a graph indicating evaluation results of the present disclosure method and the comparative method in a case where face images generated by a camera 5C are used as inputs to the mental state estimation model.

FIG. 11B is a graph indicating total evaluation results of the present disclosure method and the comparative method in a case where the face images generated by the cameras 5A to 5C are used as inputs to the mental state estimation model.

FIG. 12 is an example of functional blocks of a mental state estimation device related to training of a mental state estimation model in a second example embodiment.

FIG. 13 is an example of a flowchart related to the training of the mental state estimation model executed by the mental state estimation device in the second example embodiment.

FIG. 14 is an example of functional blocks of the mental state estimation device related to estimation of a mental state in the second example embodiment.

FIG. 15 is an example of a flowchart executed by the mental state estimation device in the second example embodiment, related to the estimation of the mental state.

FIG. 16 illustrates a schematic configuration of a mental state estimation system in a third example embodiment.

FIG. 17 is a block diagram of a mental state estimation device in a fourth example embodiment.

FIG. 18 is an example of a flowchart executed by the mental state estimation device in the fourth example embodiment.

FIG. 19 is a block diagram of a mental state estimation device in a fifth example embodiment.

FIG. 20 is an example of a flowchart executed by the mental state estimation device in the fifth example embodiment.

### EXAMPLE EMBODIMENTS

Hereinafter, example embodiments of a mental state estimation device, a mental state estimation method, and a storage medium will be described with reference to the drawings.

### First Example Embodiment

### (1) System Configuration

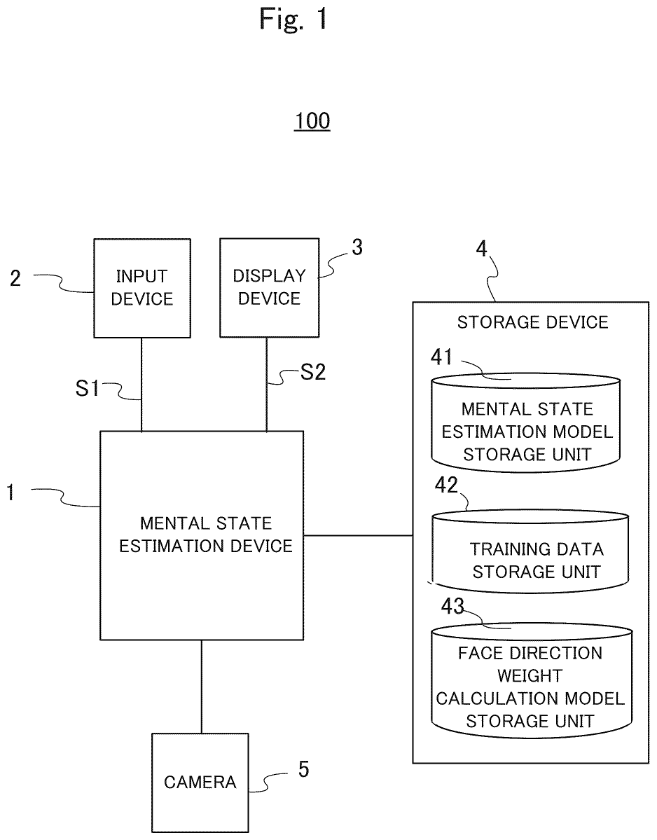

FIG. 1 illustrates a schematic configuration of a mental state estimation system 100 according to a first example embodiment. The mental state estimation system 100 is a system that estimates a mental state of a subject based on an image (face image) obtained by imaging a face of the subject, and mainly includes a mental state estimation device 1, an input device 2, a display device 3, a storage device 4, and a camera (imaging device) 5. The “subject” may be a person to be subjected to mental state estimation, and may be an athlete or an employee whose mental state is managed by an organization, or may be an individual user.

The mental state estimation device 1 estimates the mental state of the subject based on the face image (including a video as a predetermined number of images obtained in time series, and the same applies hereinafter) of the subject generated by the camera 5. The mental state estimation device 1 calculates an optional index value (score) representing the mental state of the subject as an estimation result of the mental state of the subject. Examples of the index value representing the mental state include a wakefulness level, a drowsiness level, a concentration level, a tension level, a health level, and an anxiety level. In the present example embodiment, the mental state estimation device 1 performs training of models used for estimation of the mental state (also referred to as “mental state estimation models”) before the execution of the estimation of the mental state described above. As will be described later, the mental state estimation models are a plurality of models trained using face images classified for each direction of a face. By performing the training of such mental state estimation models and using the trained mental state estimation models for the estimation of the mental state, the mental state estimation device 1 can estimate the mental state without deteriorating estimation accuracy even in a case where an installation position of the camera 5 is different between the time of the training of the models and the time of the estimation of the mental state. The training of the mental state estimation models may be performed by a device different from the mental state estimation device 1 before the estimation of the mental state by the mental state estimation device 1 is executed.

The mental state estimation device 1 performs data communication with the input device 2, the display device 3, and the camera 5 via a communication network or by wireless or wired direct communication. For example, the mental state estimation device 1 receives an input signal “S1” from the input device 2. The mental state estimation device 1 receives the face image from the camera 5 that images the face of the subject. The mental state estimation device 1 generates a display signal “S2” based on the estimation result of the mental state of the subject, and supplies the generated display signal S2 to the display device 3.

The input device 2 is an interface that receives a user input (manual input) of information related to each subject. A user who inputs the information using the input device 2 may be the subject himself/herself or a person who manages or supervises activities of the subject. The input device 2 may be, for example, various user input interfaces such as a touch panel, a button, a keyboard, a mouse, and an audio input device. The input device 2 supplies the input signal S1 generated based on the user input to the mental state estimation device 1. The display device 3 displays predetermined information based on the display signal S2 supplied from the mental state estimation device 1. Examples of the display device 3 include a display or a projector.

The storage device 4 is a memory that stores various types of information necessary for the estimation of the mental state, and the like. The storage device 4 may be an external storage device such as a hard disk connected to or incorporated in the mental state estimation device 1, or may be a storage medium such as a flash memory. The storage device 4 may be a server device that performs data communication with the mental state estimation device 1. The storage device 4 may include a plurality of devices.

The storage device 4 functionally includes a mental state estimation model storage unit 41, a training data storage unit 42, and a face direction weight calculation model storage unit 43. The mental state estimation model storage unit 41 stores parameters of the mental state estimation models. In the present example embodiment, the mental state estimation models are N models (“N” is an integer of equal to or more than 2) trained using face images classified for each face direction of the subject (that is, a direction of the face of the subject relative to the camera that has imaged the face images). The training data storage unit 42 stores training data used for the training of the mental state estimation models. The face direction weight calculation model storage unit 43 stores parameters of a face direction weight calculation model. Here, the face direction weight calculation model is a model that calculates a weight (also referred to as a “face direction weight”) for each estimation result for integrating estimation results of the mental state output by the N mental state estimation models.

Details of information stored in the mental state estimation model storage unit 41, the training data storage unit 42, and the face direction weight calculation model storage unit 43 will be described later.

The configuration of the mental state estimation system 100 illustrated in FIG. 1 is an example, and various changes may be made to the configuration. For example, the input device 2 and the display device 3 may be integrally configured. In this case, the input device 2 and the display device 3 may be configured as a tablet terminal integrated with or separated from the mental state estimation device 1. In this case, the mental state estimation device 1, the input device 2, the display device 3, and the camera 5 (and the storage device 4 may be included) may be configured as one smartphone or wearable terminal used by the subject. The mental state estimation device 1 may include a plurality of devices. In this case, the plurality of devices constituting the mental state estimation device 1 exchanges information necessary for executing processing allocated in advance between the plurality of devices.

### (2) Hardware Configuration

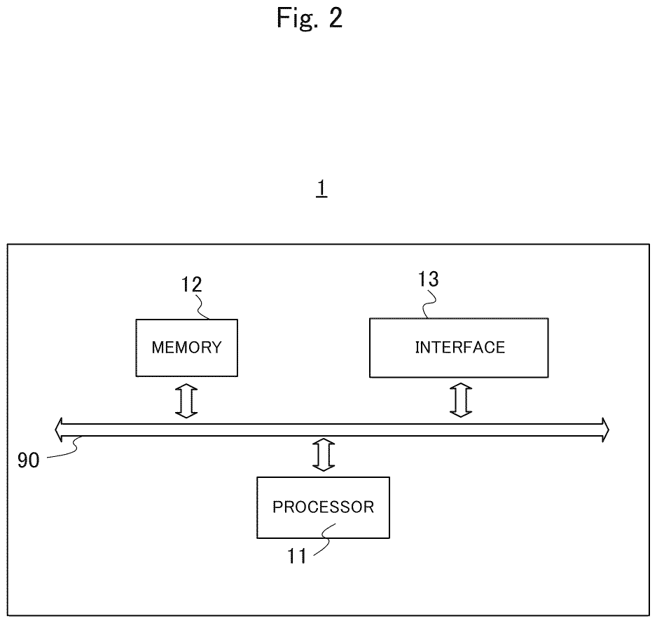

FIG. 2 illustrates a hardware configuration of the mental state estimation device 1. The mental state estimation device 1 includes, as hardware, a processor 11, a memory 12, and an interface 13. The processor 11, the memory 12, and the interface 13 are connected via a data bus 90.

The processor 11 functions as a controller (arithmetic device) that controls the entire mental state estimation device 1 by executing a program stored in the memory 12. The processor 11 is, for example, a processor such as a central processing unit (CPU), a graphics processing unit (GPU), or a tensor processing unit (TPU). The processor 11 may include a plurality of processors. The processor 11 is an example of a computer.

The memory 12 includes various volatile memories and nonvolatile memories, such as a random access memory (RAM), a read only memory (ROM), and a flash memory. The memory 12 stores a program for executing processing executed by the mental state estimation device 1. A part of information stored in the memory 12 may be stored by one or a plurality of external storage devices capable of communicating with the mental state estimation device 1, or may be stored by a storage medium detachable from the mental state estimation device 1. The memory 12 may function as at least a part of the storage device 4. In this case, the memory 12 functions as at least any one of the mental state estimation model storage unit 41, the training data storage unit 42, and the face direction weight calculation model storage unit 43.

The interface 13 is one or more interfaces for electrically connecting the mental state estimation device 1 and another device. These interfaces may be a wireless interface such as a network adapter for wirelessly transmitting and receiving data to and from the another device, or may be a hardware interface for connecting to the another device by a cable or the like.

The hardware configuration of the mental state estimation device 1 is not limited to the configuration illustrated in FIG. 2. For example, the mental state estimation device 1 may include at least one of the input device 2 and the display device 3. The mental state estimation device 1 may be connected to a sound output device such as a speaker or may incorporate such a sound output device.

### (3) Details of Data

Next, details of data stored in the mental state estimation model storage unit 41, the training data storage unit 42, and the face direction weight calculation model storage unit 43 of the storage device 4 will be described. Hereinafter, an “examinee” is a person who has become an observation target in generation of the training data, and there may be a plurality of the examinees, and the examinees may include the subject or does not have to include the subject.

The mental state estimation model storage unit 41 stores the parameters of the mental state estimation models (in other words, information necessary for constituting the mental state estimation models). In the present example embodiment, the parameters of the mental state estimation models are learned by the mental state estimation device 1 before the estimation of the mental state of the subject.

The mental state estimation models are the N models trained using face images classified into N patterns according to a direction of a face of the examinee. Hereinafter, the N mental state estimation models are referred to as a “first mental state estimation model”, . . . , and an “N-th mental state estimation model”, and the directions of the face related to the “first mental state estimation model”, . . . , and the “N-th mental state estimation model” are referred to as a “first direction”, . . . , and an “N-th direction”. Here, the “first direction”, . . . , and the “N-th direction” are the directions of the face of the different N patterns, and for example, the directions are different from each other in at least any one of a vertical direction or a horizontal direction.

In a case where “n=1, . . . , N”, an n-th mental state estimation model is a model trained based on a face image facing an n-th direction. Specifically, the n-th mental state estimation model is a model that has learned a relationship between a feature amount related to a mental state (also referred to as “mental state feature amount”) of the face image in which the face direction of the examinee is the n-th direction and a mental state of the examinee at the time of generation of the face image. In other words, the n-th mental state estimation model is trained in advance in such a way as to output the estimation result of the mental state of the person indicated in the face image in a case where the mental state feature amount calculated based on the face image in which the face direction is the n-th direction is input. Here, the mental state feature amount is a feature amount used for estimating the mental state from the face image of the subject. For example, in a case where the mental state to be estimated is drowsiness, the mental state feature amount is a value indicating an opening level of eyes. The mental state feature amount is data in a tensor format of a predetermined number of dimensions an input format to the mental state estimation models.

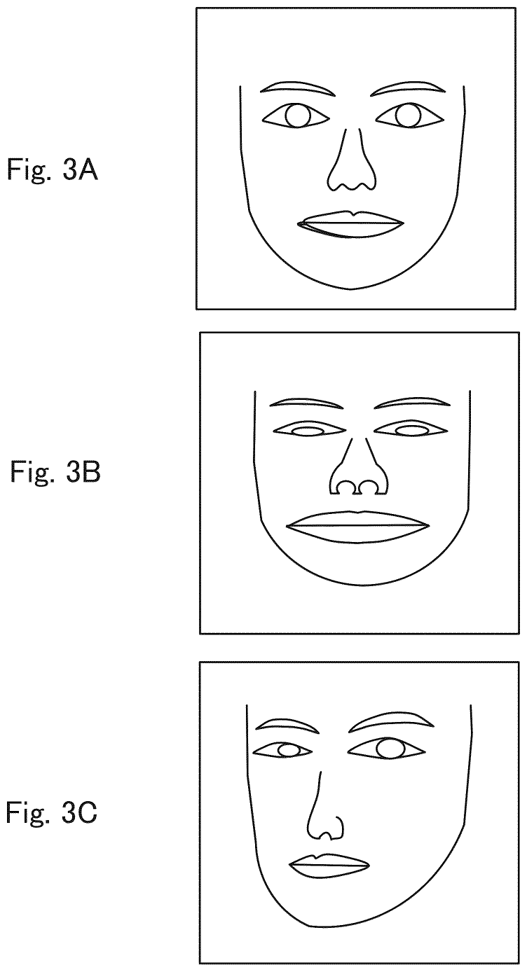

FIGS. 3A to 3C illustrate examples of face images in which directions of faces are different. The face image illustrated in FIG. 3A is a face image of a subject facing a front direction relative to the camera that performs imaging, the face image illustrated in FIG. 3B is a face image of an examinee facing upward by a predetermined angle (elevation angle) relative to the camera that performs imaging, and the face image illustrated in FIG. 3C is a face image of an examinee facing rightward by a predetermined angle relative to the camera that performs imaging. For example, in a case where the directions of the faces illustrated in FIGS. 3A to 3C are defined as the first direction to the third direction, parameters of the first mental state estimation model trained based on the face image related to the face direction illustrated in FIG. 3A, a second mental state estimation model trained based on the face image related to the face direction illustrated in FIG. 3B, and a third mental state estimation model trained based on the face image related to the face direction illustrated in FIG. 3C are stored in the mental state estimation model storage unit 41.

Each mental state estimation model may be an optional machine learning model (including a statistical model) such as a neural network or a support vector machine. For example, in a case where the mental state estimation model is a model based on the neural network such as a convolutional neural network, the mental state estimation model storage unit 41 stores information related to various parameters such as a layer structure, a neuron structure of each layer, the number of filters and a filter size in each layer, and a weight of each element of each filter. The mental state estimation models may have a common architecture or may have different architectures from each other.

The mental state estimation model may be a model trained by further classifying face images for each predetermined attribute of the examinee. In this case, the parameters of each mental state estimation model trained based on the face images classified according to the predetermined attribute and the directions of the face are stored in the mental state estimation model storage unit 41. Examples of the predetermined attribute described above include gender, job category, race, age, height, weight, muscle mass, mental state tolerance, lifestyle, exercise habit, cognitive tendency, and combinations of these.

The training data storage unit 42 stores training data used for the training of the mental state estimation models. The training data includes the face image of the examinee (for example, a time-series image of a predetermined time length) and correct answer data indicating an estimation result of a mental state that is a correct answer to be output by the mental state estimation model in a case where the face image is input to the mental state estimation model. Here, as input data to the mental state estimation model at the time of the training, the mental state estimation device 1 uses, in addition to the face images of the examinee (also referred to as “original face images”) stored in the training data storage unit 42, face images (also referred to as “augmented face images”) generated by data augmentation (data augmentation) from the face images. Here, the augmented face images are the face images of the examinee in which the directions of the face are different from the directions of the face of the examinee in the original face images, and are generated in such a way that the directions are the directions of the face insufficient in the original face images. The original face image is an example of a “first face image”, and the augmented face image is an example of a “second face image”.

The face direction weight calculation model storage unit 43 stores the parameters of the face direction weight calculation model (in other words, information necessary for constituting the face direction weight calculation model). Here, the face direction weight calculation model calculates the face direction weight in such a way that a weight to an estimation result related to a direction close to the direction of the face indicated by the input face image becomes larger. Hereinafter, in a case where “n=1, . . . , N”, an estimation result of the mental state output by the n-th mental state estimation model is also referred to as an “n-th estimation result”. The face direction weight calculation model is a model that estimates a relationship between the face image of the subject and the face direction weight according to the direction of the face of the subject.

In the present example embodiment, in a case where a feature amount calculated based on the face image (also referred to as a “face direction feature amount”) is input, the face direction weight calculation model outputs each face direction weight according to the face direction of the person indicated by the face image. The face direction feature amount is, for example, an angle representing the face direction, and may indicate a set or any one of an angle in the vertical direction and an angle in the horizontal direction of the face.

The face direction weight calculation model may be an optional model that calculates the face direction weight in such a way that the weight to the estimation result related to the direction close to the direction of the face indicated by the input face image becomes larger. For example, the face direction weight calculation model may store Gaussian distributions of representative face direction feature amounts of first to N-th estimation results, calculate a confidence interval to which the face direction feature amount input to the face direction weight calculation model belongs for each of the Gaussian distributions described above, and set the face direction weight according to the calculated confidence interval. In another example, the face direction weight calculation model may store the representative face direction feature amounts of the first to N-th estimation results, and set the face direction weight according to a distance between the representative face direction feature amounts of the first to N-th estimation results and the face direction feature amount input to the face direction weight calculation model.

In still another example, the face direction weight calculation model may be a classification model that classifies whether the direction of the face in the original face image is any one of the first to N-th directions based on the input face direction feature amount. In this case, for example, certainty factors for the first to N-th directions output by the classification model in a case where the face direction feature amount is input is set as the face direction weights for the first to N-th estimation results. In this case, the classification model may be an optional machine learning model (including a statistical model) such as a neural network or a support vector machine. For example, in a case where the mental state estimation model is the model based on the neural network such as the convolutional neural network, the face direction weight calculation model storage unit 43 stores information related to various parameters such as a layer structure, a neuron structure of each layer, the number of filters and a filter size in each layer, and a weight of each element of each filter.

In addition to the various types of information described above, the storage device 4 stores various types of information necessary for training the mental state estimation model and estimating the mental state by the mental state estimation model.

For example, the storage device 4 stores parameters of a mental state feature amount calculation model (in other words, information necessary for constituting the mental state feature amount calculation model) as a model that calculates the mental state feature amount from the face image of the subject. Similarly, the storage device 4 stores parameters of a face direction feature amount calculation model (in other words, information necessary for constituting the face direction feature amount calculation model) as a model that calculates the face direction feature amount from the face image of the subject.

Each feature amount calculation model used in the present example embodiment may be trained in such a way that the feature amount suitable for the present example embodiment is extracted. In this case, for example, each feature amount calculation model is trained using the face image prepared as the training data as the input data, and the parameters of each feature amount calculation model obtained by the training are stored in the storage device 4 in advance (that is, before the mental state estimation of the subject). Various forms have been proposed as the feature amount calculation model (feature amount extractor) using the image as the input, and a model in an optional form among the various forms may be adopted as each feature amount calculation model described above. For example, as such a feature amount calculation model, there are various deep learning models such as VGG16, VGG19, and MobileNet. For example, in a case where each feature amount calculation model described above is a model based on the neural network, the storage device 4 stores, in advance, information related to various parameters such as a layer structure, a neuron structure of each layer, the number of filters and a filter size in each layer, and a weight of each element of each filter.

### (4) Training of Mental State Estimation Models

Next, processing related to the training of the mental state estimation models will be described. Schematically, by generating the augmented face images from the original face images stored in the training data storage unit 42, the mental state estimation device 1 prepares the face images related to the first to N-th directions, and performs training of the first to N-th mental state estimation models. As a result, data augmentation of the training data is performed in such a way that an amount of the training data is sufficient for training the first to N-th mental state estimation models, and the first to N-th mental state estimation models that output highly accurate estimation results of the mental state are trained.

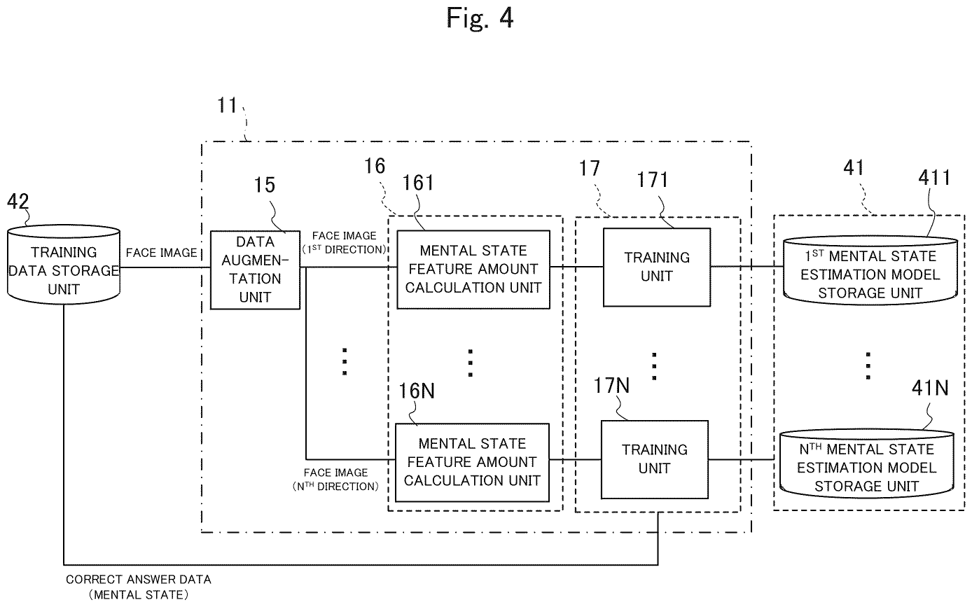

FIG. 4 is an example of functional blocks of the mental state estimation device 1 related to the training of the mental state estimation model. The processor 11 of the mental state estimation device 1 relates to the training of the mental state estimation model, and functionally includes a data augmentation unit 15, N mental state feature amount calculation units 16 (161 to 16N), and N training units 17 (171 to 17N). The mental state estimation model storage unit 41 functionally includes a first mental state estimation model storage unit 411 to an N-th mental state estimation model storage unit 41N that store parameters of the first to N-th mental state estimation models to be trained. In FIG. 4, blocks between which data is exchanged are connected by a solid line, but a combination of the blocks between which the data is exchanged is not limited to the illustrated combination. The same applies to diagrams of other functional blocks described later.

The data augmentation unit 15 acquires the original face images stored in the training data storage unit 42, and generates the augmented face images in which the directions of the face are different from those of the original face images by the data augmentation from the original face images. As a result, the data augmentation unit 15 suitably generates the face images necessary for training the N first to N-th mental state estimation models related to the first to N-th directions. In this case, the data augmentation unit 15 may convert the original face images into the augmented face images based on an optional face direction conversion technology for changing face directions of a person in images. Such a face direction conversion technology may be, for example, a method according to NPL 1. A specific example of the generation of the augmented face images by the data augmentation unit 15 will be described later. The data augmentation unit 15 supplies the face image related to the n-th direction to a mental state feature amount calculation unit 16n. As a result, the face images related to the first to N-th directions are supplied to the mental state feature amount calculation units 161 to 16N. The data augmentation unit 15 may use the original face images as they are without converting the original face images. In this case, the original face images are supplied to any of the mental state feature amount calculation units 161 to 16N according to the face directions of the person in the images.

The mental state feature amount calculation unit 16n (n=1, . . . , N) calculates a mental state feature amount from the face image related to the n-th direction. In this case, the mental state feature amount calculation unit 16n acquires the mental state feature amount output from the mental state feature amount calculation model by inputting the face image to the mental state feature amount calculation model. The mental state feature amount calculation unit 16n supplies the calculated mental state feature amount to the related training unit 17n.

The training unit 17n (n=1, . . . , N) trains the n-th mental state estimation model based on the mental state feature amount acquired from the mental state feature amount calculation unit 16n and correct answer data related to the face image used for the calculation of the mental state feature amount. In a case where the face image used to calculate the mental state feature amount is the original face image, the correct answer data described above is correct answer data stored in the training data storage unit 42 as the same record as the original face image, and in a case where the face image used to calculate the mental state feature amount is the augmented face image, the correct answer data described above is correct answer data stored in the training data storage unit 42 as the same record as the original face image used to generate the augmented face image. The training unit 17n determines parameters of the n-th mental state estimation model in such a way as to minimize an error (loss) between an estimation result of the mental state output by the n-th mental state estimation model in a case where the mental state feature amount acquired from the mental state feature amount calculation unit 16n is input to the n-th mental state estimation model and a correct answer indicated by the correct answer data. An algorithm for determining the parameters described above in such a way as to minimize the loss may be an optional training algorithm used in machine learning such as gradient descent or back propagation. The training unit 17n stores the learned parameters of the n-th mental state estimation model in an n-th mental state estimation model storage unit 41n.

The components of the data augmentation unit 15, the mental state feature amount calculation units 16, and the training units 17 described in FIG. 4 can be achieved by, for example, the processor 11 executing a program. Each component may also be achieved by recording a necessary program in an optional nonvolatile storage medium and installing the program as necessary. At least a part of these components is not limited to be achieved by software by a program, and may be achieved by a combination of any of hardware, firmware, and software, or the like. At least a part of these components may be achieved using, for example, a user-programmable integrated circuit such as a field-programmable gate array (FPGA) or a microcontroller. In this case, a program including the above components may be achieved by using the integrated circuit. At least a part of the components may include an application specific standard produce (ASSP), an application specific integrated circuit (ASIC), or a quantum processor (quantum computer control chip). In this manner, the components may be achieved by various types of hardware. The same applies to other example embodiments described later. These components may also be achieved by, for example, cooperation of a plurality of computers by using a cloud computing technology or the like.

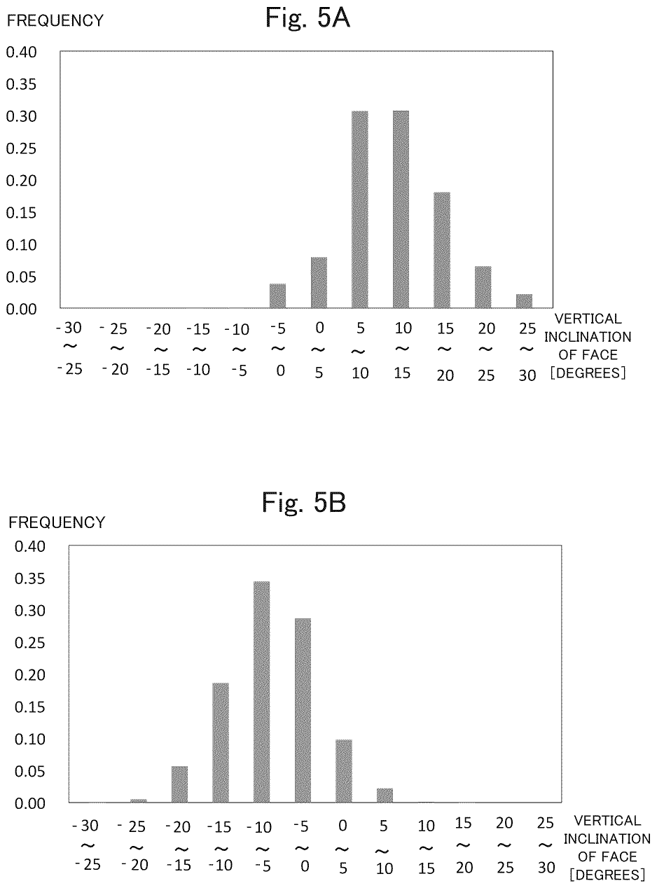

Next, the augmented face images generated by the data augmentation unit 15 will be supplementarily described. FIG. 5A illustrates a distribution related to the directions of the face in the original face images stored in the training data storage unit 42, and FIG. 5B illustrates a distribution related to the directions of the face in the augmented face images generated by the data augmentation unit 15. Here, FIGS. 5A and 5B illustrate, as an example, frequency distributions in which the face images are classified according to the directions of the face in the vertical direction. The direction of the face in the vertical direction is represented by a numerical value in which the front direction is 0 degrees, a direction in which an elevation angle increases is a positive direction, and a direction in which a depression angle increases is a negative direction, and a frequency indicates a ratio of a frequency in a case where the whole is 1.

Here, as an example, the data augmentation unit 15 generates the augmented face images in such a way that the augmented face images have the distribution in which a peak position is different from that of the distribution of the original face images. Specifically, while the distribution of the original face images illustrated in FIG. 5A is the distribution having an average value around 10 to 15 degrees, the distribution of the augmented face images illustrated in FIG. 5B is the distribution having an average value around −10 to −5 degrees. In this case, for example, the data augmentation unit 15 may generate the augmented face images in such a way as to obtain a Gaussian distribution having an average value and variance specified by a user input.

The data augmentation unit 15 does not need to generate the augmented face images in such a way as to have the Gaussian distribution, and is only required to generate the augmented face images in accordance with an optional rule in such a way that the number of samples of the face images necessary for training each of the N first to N-th mental state estimation models related to the first to N-th directions can be obtained. In the examples of FIGS. 5A and 5B, the directions of the face are classified according to the directions of the face in the vertical direction, but the present disclosure is not limited to this, and the directions of the face may be classified according to the directions of the face in the horizontal direction, or the directions of the face may be classified according to a combination of the vertical direction and the horizontal direction.

In this manner, the data augmentation unit 15 generates the augmented face images in such a way as to increase the number of samples of the face images for the directions of the face, which is insufficient only with the original face image. As a result, the data augmentation unit 15 can secure the number of samples of the face images necessary for training the N first to N-th mental state estimation models related to the first to N-th directions, and train the highly accurate first to N-th mental state estimation models.

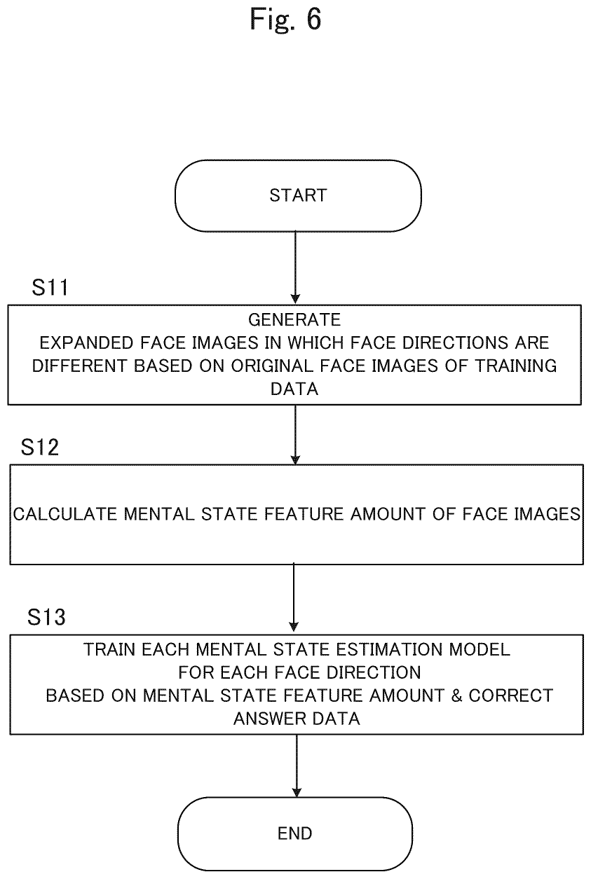

FIG. 6 is an example of a flowchart related to the training of the mental state estimation model executed by the mental state estimation device 1.

First, the mental state estimation device 1 generates augmented face images in which face directions of an examinee are different from those in original face images based on the original face images of training data stored in the training data storage unit 42 (step S11). As a result, the mental state estimation device 1 acquires the number of samples of the face images necessary for training the N first to N-th mental state estimation models related to the first to N-th directions.

Next, the mental state estimation device 1 calculates a mental state feature amount of the face images (step S12). In this case, the mental state estimation device 1 calculates the mental state feature amount to be input to the mental state estimation model in training for each sample of the face images (for example, a one-minute video).

The mental state estimation device 1 trains each mental state estimation model for each face direction based on the mental state feature amount and correct answer data (step S13). In this case, the mental state estimation device 1 updates the parameters of the n-th mental state estimation model based on the mental state feature amount of the face image related to the n-th direction and the correct answer data related to a record of the face image (the original face image in the case of the augmented face image).

### (5) Estimation of Mental State

Next, processing related to the estimation of the mental state using the trained mental state estimation models will be described. Schematically, the mental state estimation device 1 acquires estimation results of the first to N-th mental state estimation models and a face direction weight output from the face direction weight calculation model based on the face image of the subject obtained from the camera 5, and integrates the estimation results described above using the face direction weight. As a result, even in a case where the installation position of the camera 5 is different between the time of training the models and the time of estimating the mental state, it is possible to estimate the mental state without deteriorating the estimation accuracy.

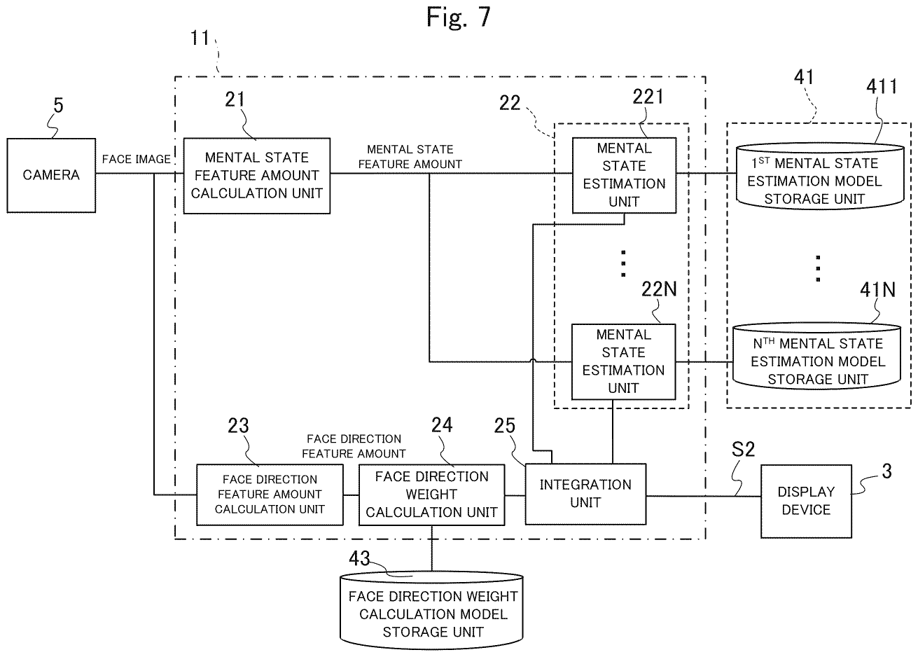

FIG. 7 is an example of functional blocks of the mental state estimation device 1 related to the estimation of the mental state using the mental state estimation model. The processor 11 of the mental state estimation device 1 relates to the estimation of the mental state using the mental state estimation model, and functionally includes a mental state feature amount calculation unit 21, N mental state estimation units 22 (221 to 22N), a face direction feature amount calculation unit 23, a face direction weight calculation unit 24, and an integration unit 25. The mental state estimation model storage unit 41 functionally includes the first mental state estimation model storage unit 411 to the N-th mental state estimation model storage unit 41N that store the parameters of the trained first to N-th mental state estimation models.

The mental state feature amount calculation unit 21 acquires the face image generated by the camera 5 via the interface 13, and calculates the mental state feature amount from the acquired face image. In this case, the mental state feature amount calculation unit 21 calculates the mental state feature amount based on the predetermined number of time-series face images (for example, one-minute video data) of the subject and the mental state feature amount calculation model. The mental state feature amount calculation model used by the mental state feature amount calculation unit 21 is the same as the mental state feature amount calculation model used by the mental state feature amount calculation unit 16n. The mental state feature amount calculation unit 21 supplies the calculated mental state feature amount to the mental state estimation units 221 to 22N.

The mental state estimation unit 22n (n=1, . . . , N) generates the n-th estimation result related to the mental state of the subject based on the n-th mental state estimation model including the parameters stored in the n-th mental state estimation model storage unit 41n and the mental state feature amount. In this case, the mental state estimation unit 22n acquires, as the n-th estimation result, an estimation result output by the n-th mental state estimation model in a case where the mental state feature amount is input to the n-th mental state estimation model. The mental state estimation unit 22n supplies the generated n-th estimation result to the integration unit 25.

The face direction feature amount calculation unit 23 calculates the face direction feature amount based on the face image acquired from the camera 5 by the mental state feature amount calculation unit 21. In this case, the face direction feature amount calculation unit 23 acquires the face direction feature amount output from the face direction feature amount calculation model in a case where the acquired face image is input to the face direction feature amount calculation model. The face direction feature amount calculation unit 23 supplies the calculated face direction feature amount to the face direction weight calculation unit 24.

The face direction weight calculation unit 24 calculates face direction weights for the first to N-th estimation results based on the face direction weight calculation model including the parameters stored in the face direction weight calculation model storage unit 43 and the face direction feature amount. The face direction weight calculation unit 24 supplies the face direction weights for the first to N-th estimation results to the integration unit 25.

The integration unit 25 generates an integrated estimation result obtained by integrating the first to N-th estimation results based on the first to N-th estimation results supplied from the mental state estimation unit 22 and the face direction weight supplied from the face direction weight calculation unit 24. In this case, for example, the integration unit 25 generates a weighted average of the first to N-th estimation results as the integrated estimation result based on the face direction weights for the first to N-th estimation results. The integration unit 25 generates the display signal S2 for displaying the generated integrated estimation result as a final estimation result of the mental state of the subject, and supplies the generated display signal S2 to the display device 3. As a result, the display device 3 displays the integrated estimation result as the final estimation result of the mental state of the subject.

The components of the mental state feature amount calculation unit 21, the mental state estimation units 22, the face direction feature amount calculation unit 23, the face direction weight calculation unit 24, and the integration unit 25 described in FIG. 7 can be achieved by, for example, the processor 11 executing a program. Each component may also be achieved by recording a necessary program in an optional nonvolatile storage medium and installing the program as necessary. At least a part of these components is not limited to be achieved by software by a program, and may be achieved by a combination of any of hardware, firmware, and software, or the like. At least a part of these components may be achieved using, for example, a user-programmable integrated circuit such as an FPGA or a microcontroller. In this case, a program including the above components may be achieved by using the integrated circuit. At least a part of the components may include an ASSP, an ASIC, or a quantum processor. In this manner, the components may be achieved by various types of hardware. The same applies to other example embodiments described later. These components may also be achieved by, for example, cooperation of a plurality of computers by using a cloud computing technology or the like.

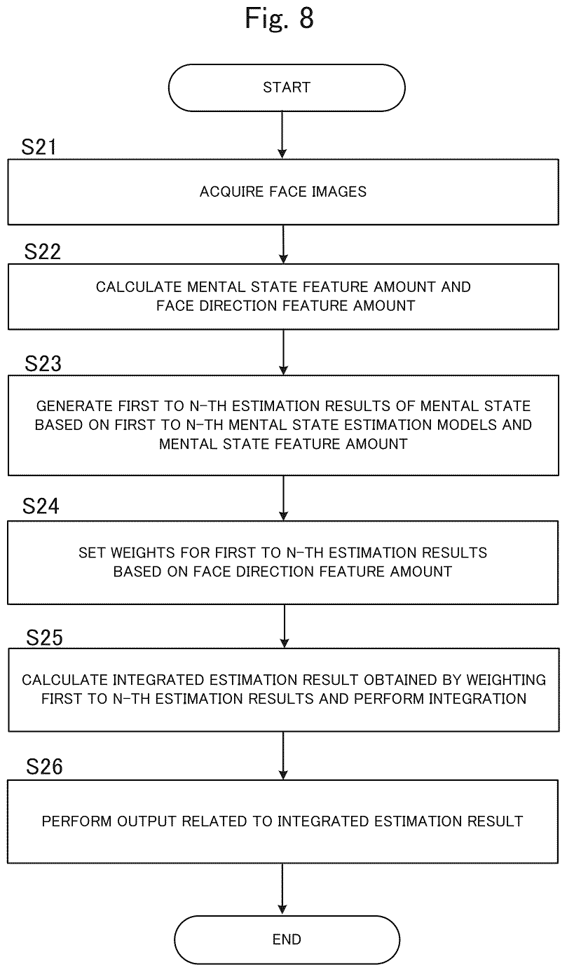

FIG. 8 is an example of a flowchart executed by the mental state estimation device 1 related to the estimation of the mental state in the first example embodiment.

First, the mental state estimation device 1 acquires face images generated by the camera 5 that images a subject (step S21). In this case, the mental state estimation device 1 acquires a predetermined number of the face images (for example, time-series images having a predetermined time length) determined in advance, which is necessary for calculating a mental state feature amount and a face direction feature amount.

The mental state estimation device 1 calculates the mental state feature amount and the face direction feature amount from the face images acquired in step S21 (step S22). In this case, the mental state estimation device 1 acquires the mental state feature amount output by the mental state feature amount calculation model in a case where the face images described above are input to the mental state feature amount calculation model, and acquires the face direction feature amount output by the face direction feature amount calculation model in a case where the face images described above are input to the face direction feature amount calculation model.

Next, the mental state estimation device 1 generates the first to N-th estimation results of a mental state of the subject based on the first to N-th mental state estimation models configured with reference to the mental state estimation model storage unit 41 and the mental state feature amount calculated in step S22 (step S23). In this case, the mental state estimation device 1 acquires the first to N-th estimation results from the first to N-th mental state estimation models by inputting the mental state feature amount to each of the first to N-th mental state estimation models.

Next, the mental state estimation device 1 sets weights for the first to N-th estimation results based on the face direction feature amount calculated in step S22 (step S24). In this case, the mental state estimation device 1 calculates the face direction weights for the first to N-th estimation results based on the face direction weight calculation model configured with reference to the face direction weight calculation model storage unit 43 and the face direction feature amount. Steps S23 and S24 are in no particular order, and may be performed in the reverse order or may be executed substantially simultaneously by parallel processing.

Next, the mental state estimation device 1 calculates an integrated estimation result obtained by weighting the first to N-th estimation results with the face direction weights and performing integration (step S25). The mental state estimation device 1 performs an output related to the calculated integrated estimation result (step S26). In this case, as a final estimation result of the mental state of the subject, the mental state estimation device 1 may display the integrated estimation result on the display device 3, or output the integrated estimation result by audio by an audio output device (not illustrated), may store the integrated estimation result in the storage device 4 or the like, or may transmit the integrated estimation result to another device.

### (6) Example

The applicant recorded face images of a subject during a calculation task with three cameras, constructed the recorded face images and correct answer data indicating a correct mental state of the subject at the time of recording, which is generated by a questionnaire result for the target, measurement by a sensor, or the like, as an evaluation data set, and evaluated a method of estimating the mental state based on the present example embodiment (also referred to as “the present disclosure method”). Here, the mental state to be estimated is assumed to be a wakefulness level. In order to verify effectiveness of the present disclosure method, a method of estimating the mental state using one mental state estimation model trained regardless of a direction of a face (also referred to as a “comparative method”) was also evaluated. In the comparative method, the mental state estimation model is trained using the original faces image having the distribution illustrated in FIG. 5A generated by one camera, and in the present disclosure method, the first mental state estimation model and the second mental state estimation model are trained with “N=2”, the first mental state estimation model is trained using the original face images having the distribution illustrated in FIG. 5A generated by one camera, and the second mental state estimation model is trained using the augmented face images having the distribution illustrated in FIG. 5B.

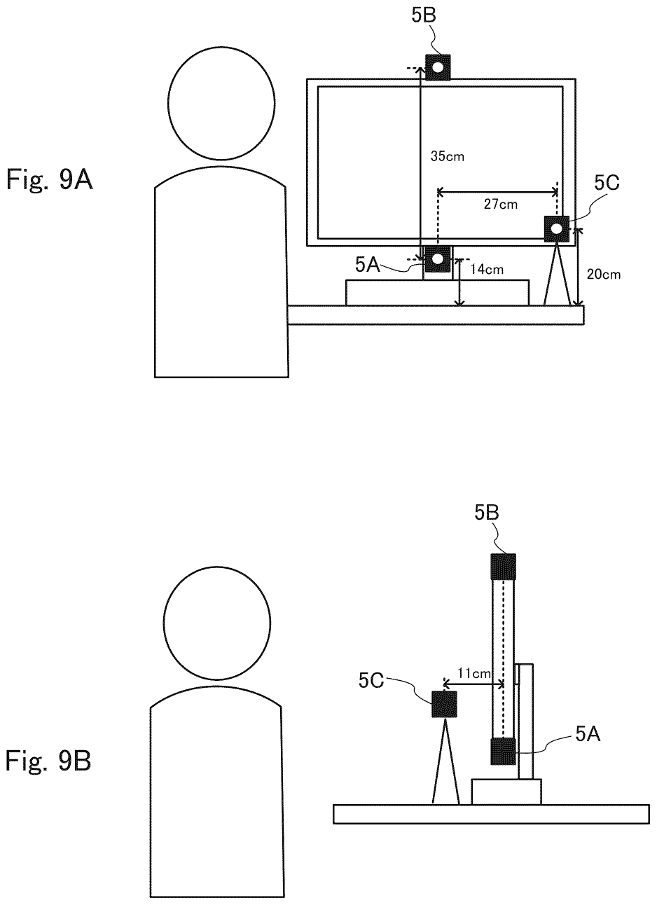

FIG. 9A is a diagram of a generation environment of the evaluation data set observed from a direction in which the subject exists, and FIG. 9B is a diagram of the generation environment of the evaluation data set observed from a side of the subject. As illustrated in FIG. 9A, cameras 5A to 5C are installed at different heights, and image the same subject with different inclinations of a face. As illustrated in FIGS. 9A and 9B, the camera 5C is installed at a position shifted from positions of the cameras 5A and 5B in the horizontal direction and a depth direction. Here, the number of subjects is 27 (24 males and 3 females) in total, the subject executes the calculation task displayed on a display for 15 minutes, and the face images of the subject during the execution of the calculation task are generated from the cameras 5A to 5C as the evaluation data set. A length of the face images per sample (that is, the length of the face images included in one record) is assumed to be one minute. The evaluation data sets were tabulated by classifying the evaluation data sets into four according to whether the inclination in the vertical direction of the face belongs to “−30 to −15”, “−15 to 0”, “0 to 15”, or “15 to 30”.

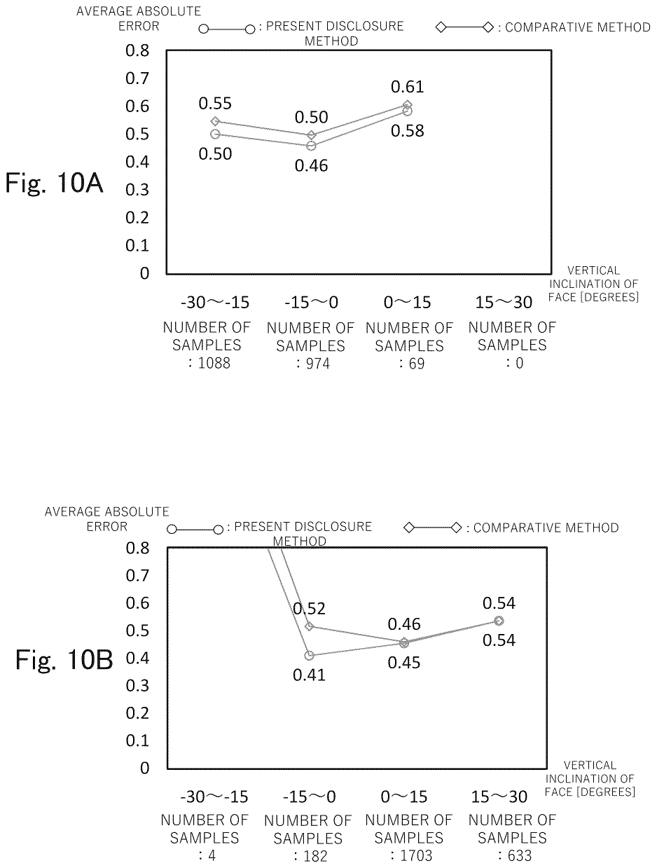

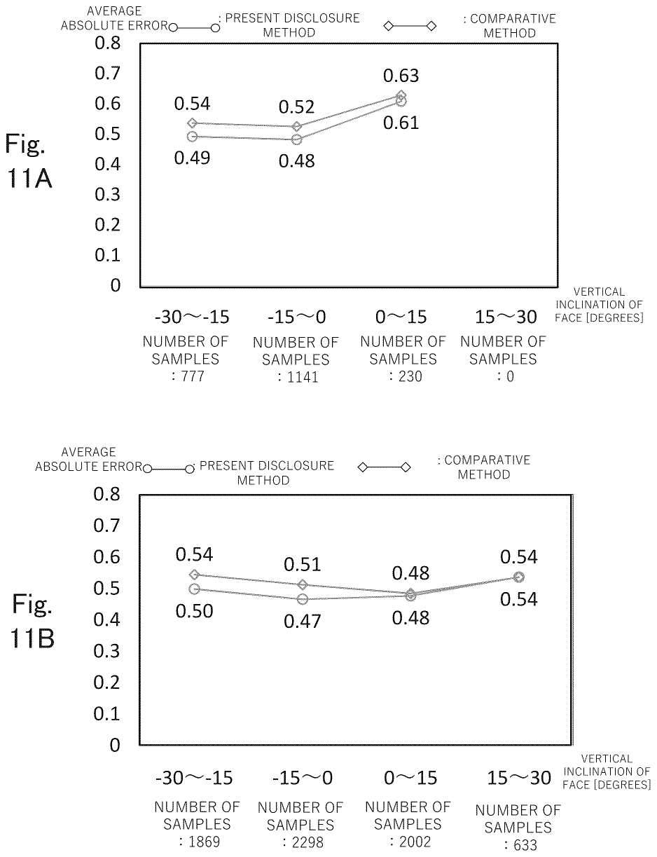

FIG. 10A is a graph indicating evaluation results of the present disclosure method and the comparative method in a case where the face images generated by the camera 5A are used as inputs to the mental state estimation model. FIG. 10B is a graph indicating evaluation results of the present disclosure method and the comparative method in a case where the face images generated by the camera 5B are used as inputs to the mental state estimation model. FIG. 11A is a graph indicating evaluation results of the present disclosure method and the comparative method in a case where the face images generated by the camera 5C are used as inputs to the mental state estimation model. FIG. 11B is a graph indicating total evaluation results of the present disclosure method and the comparative method in a case where the face images generated by the cameras 5A to 5C are used as inputs to the mental state estimation model. In each of FIGS. 10A to 11B, a vertical axis represents an average absolute error (value normalized in such a way that a value range is equal to or less than 5.0) between a wakefulness level of a correct answer indicated by the correct answer data and an estimated value of the wakefulness level obtained by the present disclosure method or the comparative method, and a horizontal axis represents the inclination in the vertical direction of the face of the subject related to “−30 to −15”, “15 to 0”, “0 to 15”, or “15 to 30”. To the horizontal axis, the number of samples (that is, the number of records) of the face images obtained for a range of the directions of the face of each subject is also added.

As illustrated in FIGS. 10A to 11B, in the face images generated by all of the cameras 5A to 5C, an error increase in a case where the inclination in the vertical direction of the face is a negative value (that is, in a case where the face is imaged from below) is suppressed in the present disclosure method as compared with that in the comparative method. In this manner, in the present disclosure method, robustness for the position of the camera 5 that performs imaging is improved.

### Second Example Embodiment

A second example embodiment is different from the first example embodiment in that a mental state estimation device 1 uses, as a mental state estimation model, a model that uses, as an input, a set of a mental state feature amount and a face direction feature amount obtained from a face image and outputs an estimation result of a mental state in consideration of a face direction in the face image. In other words, the mental state estimation model in the second example embodiment is the model that has learned a relationship between the set of the mental state feature amount and the face direction feature amount calculated from the face image and a mental state of a subject at the time of generation of the face image. Hereinafter, the same components as those of the first example embodiment are appropriately denoted by the same reference signs, and description thereof will be omitted.

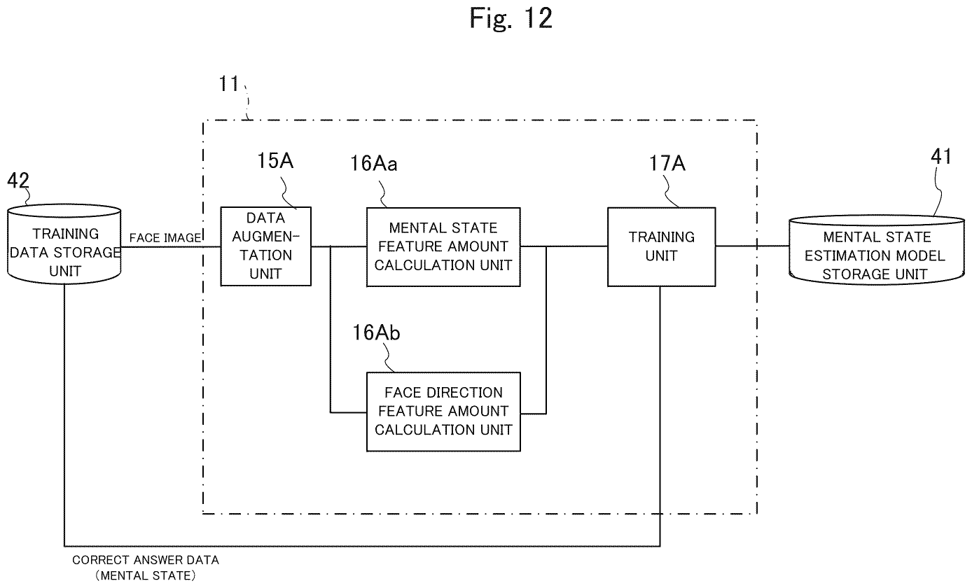

FIG. 12 is an example of functional blocks of the mental state estimation device 1 related to training of the mental state estimation model in the second example embodiment. A processor 11 of the mental state estimation device 1 in the second example embodiment relates to the training of the mental state estimation model, and functionally includes a data augmentation unit 15A, a mental state feature amount calculation unit 16Aa, a face direction feature amount calculation unit 16Ab, and a training unit 17A.

The data augmentation unit 15A acquires original face images stored in a training data storage unit 42, and generates augmented face images in which directions of the face are different from those of the original face images by data augmentation from the original face images. As a result, the data augmentation unit 15A increases variations in the face directions of the face images used for training the mental state estimation model, and improves estimation accuracy of the mental state estimation model to be trained. The data augmentation unit 15A supplies the face images (the original face images or the augmented face images) for each sample to the mental state feature amount calculation unit 16Aa and the face direction feature amount calculation unit 16Ab.

The mental state feature amount calculation unit 16Aa calculates the mental state feature amount from the face image supplied from the data augmentation unit 15A. The mental state feature amount calculation unit 16Aa acquires the mental state feature amount output from a mental state feature amount calculation model by inputting the face image to the mental state feature amount calculation model. The mental state feature amount calculation unit 16Aa supplies the calculated mental state feature amount to the training unit 17A.

The face direction feature amount calculation unit 16Ab calculates the face direction feature amount from the face image supplied from the data augmentation unit 15A. The face direction feature amount calculation unit 16Ab acquires the face direction feature amount output from a face direction feature amount calculation model by inputting the face image to a face direction feature amount calculation model. The face direction feature amount calculation unit 16Ab supplies the calculated face direction feature amount to the training unit 17A. Parameters of the mental state feature amount calculation model and the face direction feature amount calculation model are stored in advance in a storage device 4, for example, as in the first example embodiment.

The training unit 17A trains the mental state estimation model based on the mental state feature amount acquired from the mental state feature amount calculation unit 16Aa, the face direction feature amount acquired from the face direction feature amount calculation unit 16Ab, and correct answer data related to the face image used to calculate the mental state feature amount and the face direction feature amount. In a case where the face image used to calculate the mental state feature amount is the original face image, the correct answer data described above is the correct answer data stored in the training data storage unit 42 as the same record as the original face image, and in a case where the face image used to calculate the mental state feature amount is the augmented face image, the correct answer data described above is correct answer data stored in the training data storage unit 42 as the same record as the original face image used to generate the augmented face image. The training unit 17A determines parameters of the mental state estimation model in such a way as to minimize an error (loss) between the estimation result of the mental state output by the mental state estimation model in a case where the set of the mental state feature amount and the face direction feature amount is input to the mental state estimation model and a correct answer indicated by the correct answer data. The training unit 17A stores the learned parameters of the mental state estimation model in a mental state estimation model storage unit 41.

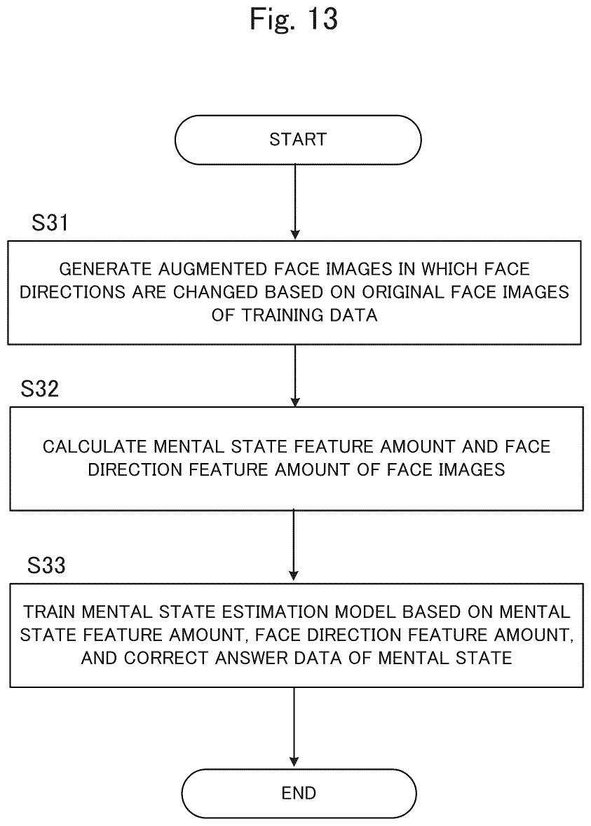

FIG. 13 is an example of a flowchart related to the training of the mental state estimation model executed by the mental state estimation device 1 in the second example embodiment.

First, the mental state estimation device 1 generates augmented face images in which face directions of an examinee is changed based on original face images of training data stored in the training data storage unit 42 (step S31).

Next, the mental state estimation device 1 calculates a mental state feature amount and a face direction feature amount of the face images (step S32). In this case, the mental state estimation device 1 calculates the mental state feature amount and the face direction feature amount to be input to the mental state estimation model for each sample of the face images (for example, a one-minute video).

The mental state estimation device 1 trains the mental state estimation model based on the mental state feature amount, the face direction feature amount, and correct answer data (step S33). In this case, the mental state estimation device 1 updates the parameters of the mental state estimation model based on a set of the mental state feature amount and the face direction feature amount and the correct answer data related to a record of the face image (an original face image in the case of an augmented face image) used to calculate the mental state feature amount and the face direction feature amount.

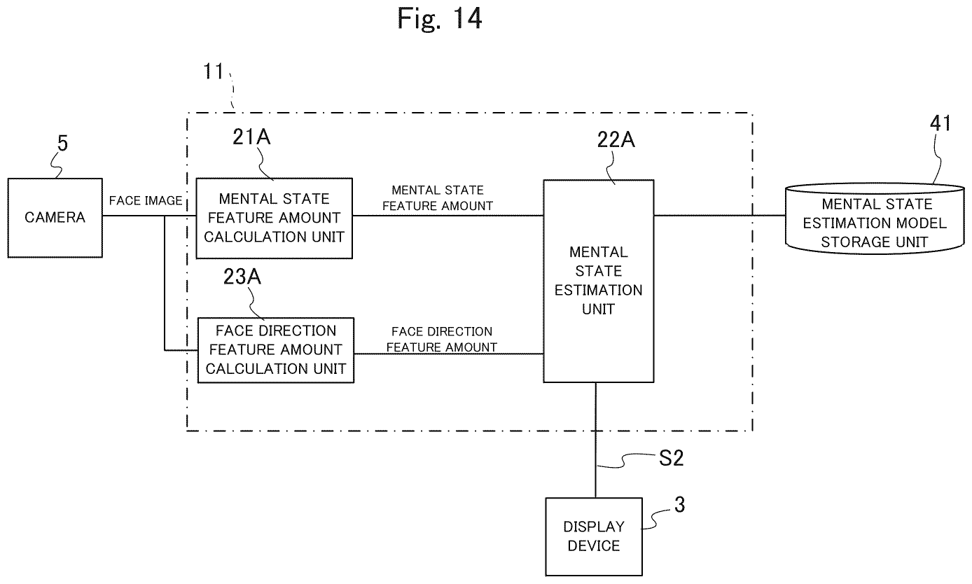

FIG. 14 is an example of functional blocks of the mental state estimation device 1 related to the estimation of the mental state using the mental state estimation model in the second example embodiment. The processor 11 of the mental state estimation device 1 in the second example embodiment relates to the estimation of the mental state using the mental state estimation model, and functionally includes a mental state feature amount calculation unit 21A, a face direction feature amount calculation unit 23A, and a mental state estimation unit 22A.

The mental state feature amount calculation unit 21 A acquires a face image generated by a camera 5 via an interface 13, and calculates a mental state feature amount from the acquired face image. In this case, the mental state feature amount calculation unit 21A calculates the mental state feature amount based on the mental state feature amount calculation model from the predetermined number of time-series face images (for example, one-minute video data) of a subject. The mental state feature amount calculation unit 21A supplies the calculated mental state feature amount to the mental state estimation unit 22A.

The face direction feature amount calculation unit 23A calculates a face direction feature amount based on the face image acquired from the camera 5 by the mental state feature amount calculation unit 21A. In this case, the face direction feature amount calculation unit 23A acquires the face direction feature amount output from the face direction feature amount calculation model in a case where the face image is input to the face direction feature amount calculation model. The face direction feature amount calculation unit 23A supplies the calculated face direction feature amount to the mental state estimation unit 22A.

The mental state estimation unit 22A generates an estimation result related to a mental state of the subject based on the mental state estimation model including the learned parameters stored in the mental state estimation model storage unit 41, the mental state feature amount calculated by the mental state feature amount calculation unit 21A, and the face direction feature amount calculated by the face direction feature amount calculation unit 23A. In this case, the mental state estimation unit 22A acquires the estimation result output by the mental state estimation model in a case where a set of the mental state feature amount and the face direction feature amount is input to the mental state estimation model. The mental state estimation unit 22A generates a display signal S2 for displaying the generated estimation result as a final estimation result of the mental state of the subject, and supplies the generated display signal S2 to a display device 3. As a result, the display device 3 displays the estimation result of the mental state of the subject.

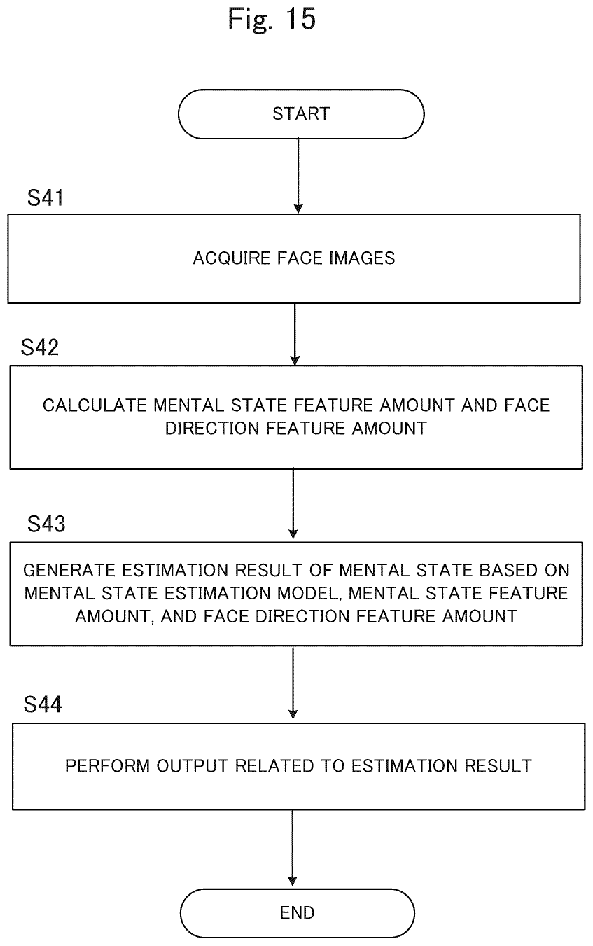

FIG. 15 is an example of a flowchart executed by the mental state estimation device 1 in the second example embodiment, related to the estimation of the mental state using the mental state estimation model.

First, the mental state estimation device 1 acquires face images generated by the camera 5 that images a subject (step S41). In this case, the mental state estimation device 1 acquires a predetermined number of face images necessary for calculating a mental state feature amount and a face direction feature amount.

The mental state estimation device 1 calculates the mental state feature amount and the face direction feature amount from the face images acquired in step S41 (step S42). In this case, the mental state estimation device 1 acquires the mental state feature amount output by the mental state feature amount calculation model in a case where the face images described above are input to the mental state feature amount calculation model, and acquires the face direction feature amount output by the face direction feature amount calculation model in a case where the face images described above are input to the face direction feature amount calculation model.

Next, the mental state estimation device 1 generates an estimation result of a mental state of the subject based on the mental state estimation model configured with reference to the mental state estimation model storage unit 41 and a set of the mental state feature amount and the face direction feature amount calculated in step S42 (step S43). In this case, the mental state estimation device 1 acquires the estimation result of the mental state from the mental state estimation model by inputting the set of the mental state feature amount and the face direction feature amount to the mental state estimation model.

The mental state estimation device 1 performs an output related to the calculated estimation result (step S44). In this case, the mental state estimation device 1 may display the estimation result of the mental state on the display device 3 or output the estimation result of the mental state by audio by an audio output device (not illustrated), may store the estimation result of the mental state in the storage device 4 or the like, or may transmit the estimation result of the mental state to another device.

Similarly to the mental state estimation device 1 in the first example embodiment, even in a case where an installation position of the camera 5 is different between a time of training the model and a time of estimating the mental state, the mental state estimation device 1 in the second example embodiment can estimate the mental state without deteriorating the estimation accuracy.

### Third Example Embodiment

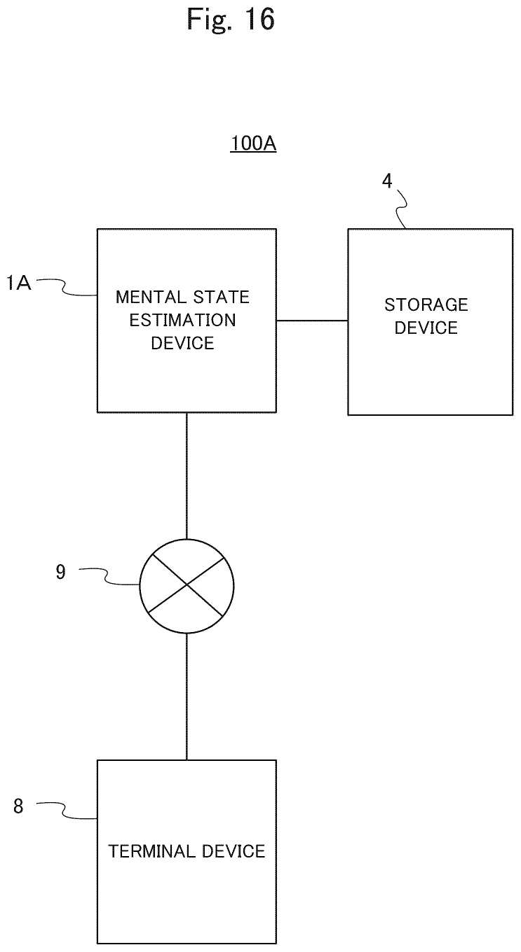

FIG. 16 illustrates a schematic configuration of a mental state estimation system 100A in a third example embodiment. The mental state estimation system 100A according to the third example embodiment includes a mental state estimation device 1A that performs the same processing as that of the mental state estimation device 1 of the first or second example embodiment, a storage device 4, and a terminal device 8 used by a subject. Hereinafter, the same components as those of the first example embodiment are appropriately denoted by the same reference signs, and description thereof will be omitted.

In the third example embodiment, the mental state estimation device 1A functions as a server, and the terminal device 8 functions as a client. The mental state estimation device 1A and the terminal device 8 perform data communication via a network 9.

The terminal device 8 is a terminal used by a user as the subject, has an input function, a display function, a communication function, and an imaging function, and functions as the input device 2, the display device 3, the camera 5, and the like illustrated in FIG. 1. The terminal device 8 may be, for example, a tablet terminal such as a personal computer or a smartphone, or a personal digital assistant (PDA). The terminal device 8 is electrically connected to the camera 5 such as a wearable sensor worn by the user, and transmits a face image of the subject output by the camera 5 to the mental state estimation device 1A via the network 9.

The mental state estimation device 1A has the same hardware configuration as the hardware configuration of the mental state estimation device 1 illustrated in FIG. 2, and a processor 11 of the mental state estimation device 1A has the functional blocks described in the first or second example embodiment. The mental state estimation device 1A receives the face image from the terminal device 8 via the network 9, and executes processing of estimating a mental state of the subject with reference to various types of information stored in the storage device 4. The mental state estimation device 1A transmits an output signal for outputting a mental state estimation result to the terminal device 8 via the network 9 based on a display request from the terminal device 8. The mental state estimation device 1A may perform processing related to training of the mental state estimation model in the first or second example embodiment based on training data stored in the storage device 4.

In this manner, the mental state estimation device 1A in the third example embodiment estimates the mental state of the subject as the user of the terminal device 8, and can more suitably present the estimation result to the subject by the terminal device 8.

### Fourth Example Embodiment

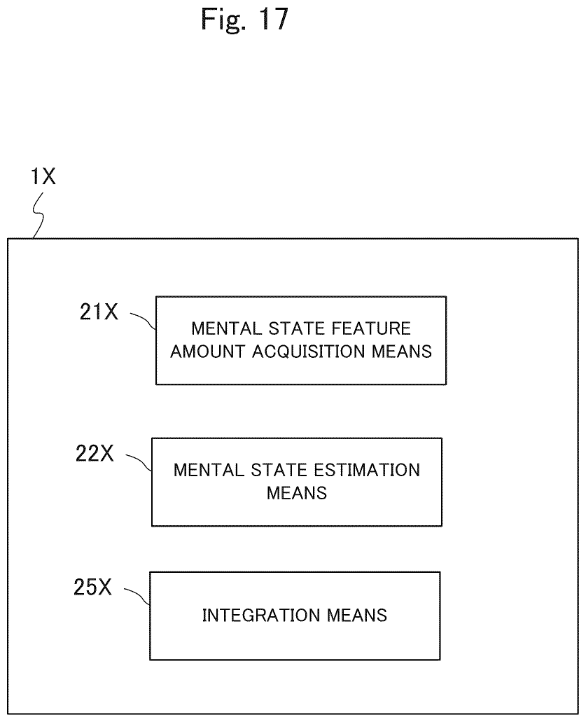

FIG. 17 illustrates a block diagram of the mental state estimation device 1X according to the fourth example embodiment. The mental state estimation device 1X mainly includes a mental state feature amount acquisition means 21X, a mental state estimation means 22X, and an integration means 25X. The mental state estimation device 1X may be configured by a plurality of devices.

The mental state feature amount acquisition means 21X is configured to acquire a mental state feature amount as a feature amount calculated from a face image of a subject and used for estimation of a mental state of the subject. The face image may be a single image or may be a predetermined number of images more than one. The mental state feature amount acquisition means 21X may be configured to acquire the mental state feature amount by calculating the mental state feature amount based on the face image of the subject or may acquire the mental state feature amount by receiving the mental state feature amount from a device, which calculates the mental state feature amount, other than the mental state estimation device 1X. Examples of the mental state feature acquisition means 21X include the mental state feature amount calculation unit 21 according to the first example embodiment or the third example embodiment.

The mental state estimation means 22X is configured to acquire a predetermined number of estimation results of the mental state based on the mental state feature amount and a predetermined number of mental state estimation models each trained using face images in which face directions are different. Examples of the mental state estimation means 22X include the mental state estimation unit 22 according to the first example embodiment or the third example embodiment.

The integration means is configured to generate an integrated estimation result obtained by integrating the predetermined number of the estimation results of the mental state. The integration means may generate the integrated estimation result as a weighted average of the predetermined number of the mental state estimation results, or may generate the integrated estimation result as a representative value such as an average of the predetermined number of the mental state estimation results. Examples of the integration means include the integration unit 25 according to the first example embodiment or the third example embodiment.

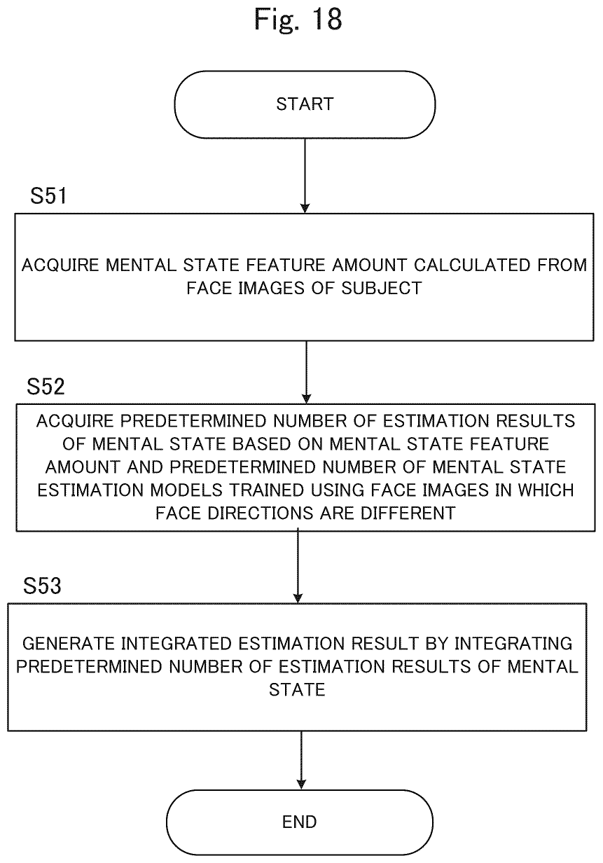

FIG. 18 is an example of a flowchart executed by the mental state estimation device 1X according to the fourth example embodiment. The mental state feature amount acquisition means 21X acquires a mental state feature amount as a feature amount calculated from a face image of a subject and used for estimation of a mental state of the subject (step S51). Next, the mental state estimation means 22X acquires a predetermined number of estimation results of the mental state based on the mental state feature amount and a predetermined number of mental state estimation models each trained using face images in which face directions are different (step S52). The integration means generates an integrated estimation result obtained by integrating the predetermined number of the estimation results of the mental state (step S53).

According to the fourth example embodiment, the mental state estimation device 1X can estimate the mental state of the subject with high accuracy.

### Fifth Example Embodiment

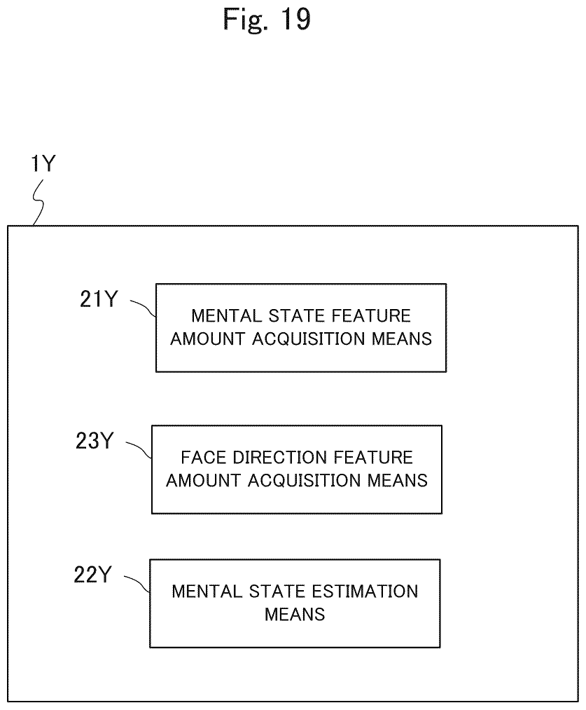

FIG. 19 is a block diagram of the mental state estimation device 1Y according to the fifth example embodiment. The mental state estimation device 1Y mainly include a mental state feature amount acquisition means 21Y, a face direction feature amount acquisition means 23Y, and a mental state estimation means 22Y. The mental state estimation device 1Y may be configured by a plurality of devices.

The mental state feature amount acquisition means 21Y is configured to acquire a mental state feature amount as a feature amount calculated from a face image of a subject and used for estimation of a mental state of the subject. The face image may be a single image or may be a predetermined number of images more than one. The mental state feature amount acquisition means 21Y may be configured to acquire the mental state feature amount by calculating the mental state feature amount based on the face image of the subject or may acquire the mental state feature amount by receiving the mental state feature amount from a device, which calculates the mental state feature amount, other than the mental state estimation device 1Y. Examples of the mental state feature acquisition means 21Y include the mental state feature amount calculation unit 21A according to the second example embodiment or the third example embodiment.

The face direction feature amount acquisition means is configured to acquire a face direction feature amount as a feature amount related to a face direction of the subject and calculated from the face image. The face direction feature amount acquisition means may acquire the face direction feature amount by calculating the one from the face image of the subject or may acquire the face direction feature amount by receiving the one from a device which calculates the face direction feature amount other than the mental state estimation device 1Y. Examples of the face direction feature amount acquisition means 23Y include the face direction feature amount acquisition means 23A according to the second example embodiment or the third example embodiment.

The mental state estimation means 22Y is configured to estimate the mental state based on the mental state feature amount and the face direction feature amount. Examples of the mental state estimation means 22Y include the mental state estimation unit 22A according to the second example embodiment or the third example embodiment.

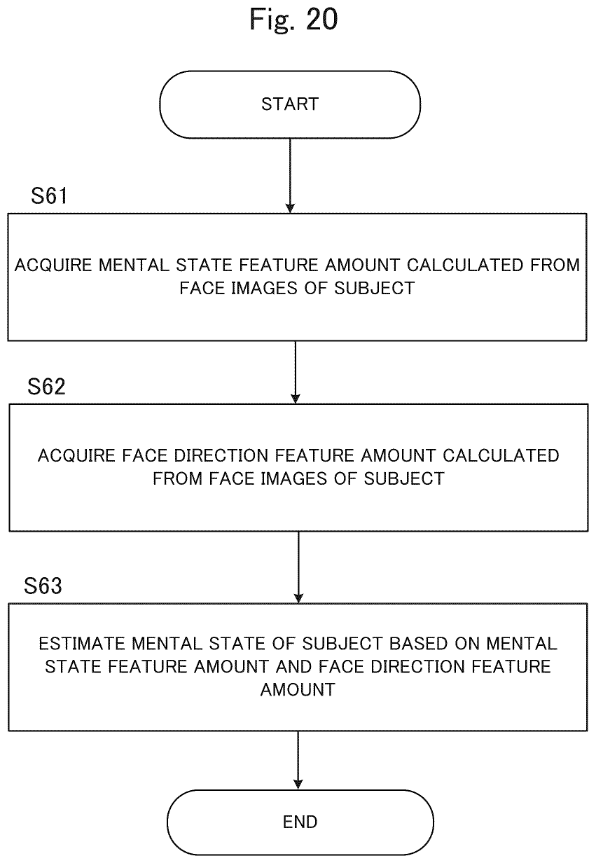

FIG. 20 illustrates an example of a flowchart executed by the mental state estimation device 1Y according to the fifth example embodiment.

The mental state feature amount acquisition means 21Y acquires a mental state feature amount as a feature amount calculated from a face image of a subject and used for estimation of a mental state of the subject (step S61). The face direction feature amount acquisition means acquires a face direction feature amount as a feature amount related to a face direction of the subject and calculated from the face image (step S62). The mental state estimation means 22Y estimates the mental state based on the mental state feature amount and the face direction feature amount (step S63).

According to the fifth example embodiment, the mental state estimation device 1Y can estimate the mental state of the subject with high accuracy.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments (including modifications, the same shall apply hereinafter) described above can be described as, but not limited to, the following Supplementary Notes.

### [Supplementary Note 1]

A mental state estimation device comprising:
a mental state feature amount acquisition means for acquiring a mental state feature amount as a feature amount calculated from a face image of a subject and used for estimation of a mental state of the subject;
a mental state estimation means for acquiring a predetermined number of estimation results of the mental state based on the mental state feature amount and a predetermined number of mental state estimation models each trained using face images in which face directions are different; and
an integration means for generating an integrated estimation result obtained by integrating the predetermined number of the estimation results of the mental state.

### [Supplementary Note 2]

The mental state estimation device according to Supplementary Note 1, wherein each of the predetermined number of the mental state estimation models is a model that has learned a relationship between the mental state feature amount of the face image in which the face direction is associated with the mental state estimation model and the mental state at a time of generation of the face image.

### [Supplementary Note]3

The mental state estimation device according to Supplementary Note 1, wherein the predetermined number of the mental state estimation models are trained using a first face image as a face image obtained by imaging an examinee and a second face image obtained by converting the first face image in such a way that a face direction of the examinee is different from a face direction in the first face image.

### [Supplementary Note 4]

The mental state estimation device according to Supplementary Note 1, further comprising
a weight determination means for determining a weight to be set to each of the predetermined number of the estimation results of the mental state based on the face image,
wherein the integration means generates the integrated estimation result based on the weight and the predetermined number of the estimation results of the mental state.

### [Supplementary Note 5]

The mental state estimation device according to Supplementary Note 4, further comprising
a face direction feature amount acquisition means for acquiring a face direction feature amount as a feature amount related to a face direction of the subject and calculated from the face image,
wherein the weight determination means determines the weight based on the face direction feature amount.

### [Supplementary Note 6]

A mental state estimation device comprising:
a mental state feature amount acquisition means for acquiring a mental state feature amount as a feature amount calculated from a face image of a subject and used for estimation of a mental state of the subject;
a face direction feature amount acquisition means for acquiring a face direction feature amount as a feature amount related to a face direction of the subject and calculated from the face image; and
a mental state estimation means for estimating the mental state based on the mental state feature amount and the face direction feature amount.

### [Supplementary Note 7]

The mental state estimation device according to Supplementary Note 6, wherein the mental state estimation means estimates the mental state based on the mental state feature amount, the face direction feature amount, and a mental state estimation model, and the mental state estimation model is a model that has learned a relationship between a set of the mental state feature amount and the face direction feature amount calculated from the face image and the mental state at a time of generation of the face image.

### [Supplementary Note 8]

A mental state estimation method by a computer, the mental state estimation method comprising:
acquiring a mental state feature amount as a feature amount calculated from a face image of a subject and used for estimation of a mental state of the subject;
acquiring a predetermined number of estimation results of the mental state based on the mental state feature amount and a predetermined number of mental state estimation models each trained using face images in which face directions are different; and
generating an integrated estimation result obtained by integrating the predetermined number of the estimation results of the mental state.

### [Supplementary Note 9]

A mental state estimation method by a computer, the mental state estimation method comprising:
acquiring a mental state feature amount as a feature amount calculated from a face image of a subject and used for estimating a mental state of the subject;
acquiring a face direction feature amount as a feature amount related to a face direction of the subject and calculated from the face image; and estimating the mental state based on the mental state feature amount and the face direction feature amount.

### [Supplementary Note 10]

A storage medium storing a program for causing a computer to execute processing comprising:
acquiring a mental state feature amount as a feature amount calculated from a face image of a subject and used for estimation of a mental state of the subject;
acquiring a predetermined number of estimation results of the mental state based on the mental state feature amount and a predetermined number of mental state estimation models each trained using face images in which face directions are different; and generating an integrated estimation result obtained by integrating the predetermined number of the estimation results of the mental state.

### [Supplementary Note 11]

A storage medium storing a program for causing a computer to execute processing comprising:
acquiring a mental state feature amount as a feature amount calculated from a face image of a subject and used for estimating a mental state of the subject;
acquiring a face direction feature amount as a feature amount related to a face direction of the subject and calculated from the face image; and
estimating the mental state based on the mental state feature amount and the face direction feature amount.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1, 1A, 1X, 1Y Mental state estimation device
2 Input device
3 Display device
4 Sorage device
5, 5A to 5C Camera
8 Terminal device
9 Network
11 Processor
12 Memory
13 Interface
41 Mental state estimation model storage unit
42 Training data storage unit
43 Face direction weight calculation model storage unit
90 Data bus
100,100A Mental state estimation system

What is claimed is:

1. A mental state estimation device comprising:
at least one memory configured to store instructions; and
at least one processor configured to execute the instructions to:
acquire a mental state feature amount as a feature amount calculated from a face image of a subject and used for estimation of a mental state of the subject;
acquire a predetermined number of estimation results of the mental state based on the mental state feature amount and a predetermined number of mental state estimation models each trained using face images in which face directions are different,
wherein each of the predetermined number of the mental state estimation models is a model that has learned by machine learning a relationship between the mental state feature amount of the face image in which the face direction is associated with the mental state estimation model and the mental state at a time of generation of the face image; and
generate an integrated estimation result obtained by integrating the predetermined number of the estimation results of the mental state.

2. (canceled)

3. The mental state estimation device according to claim 1, wherein the predetermined number of the mental state estimation models are trained using a first face image as a face image obtained by imaging an examinee and a second face image obtained by converting the first face image in such a way that a face direction of the examinee is different from a face direction in the first face image.

4. The mental state estimation device according to claim 1,
wherein the at least one processor is configured to executee the instructions to
determine a weight to be set to each of the predetermined number of the estimation results of the mental state based on the face image, and
generate the integrated estimation result based on the weight and the predetermined number of the estimation results of the mental state.

5. The mental state estimation device according to claim 4,
wherein the at least one processor is configured to execute the instructions to
acquire a face direction feature amount as a feature amount related to a face direction of the subject and calculated from the face image, and
determine the weight based on the face direction feature amount.

6. (canceled)

7. (canceled)

8. A mental state estimation method by a computer, the mental state estimation method comprising:
acquiring a mental state feature amount as a feature amount calculated from a face image of a subject and used for estimation of a mental state of the subject;
acquiring a predetermined number of estimation results of the mental state based on the mental state feature amount and a predetermined number of mental state estimation models each trained using face images in which face directions are different,
wherein each of the predetermined number of the mental state estimation models is a model that has learned a relationship between the mental state feature amount of the face image in which the face direction is associated with the mental state estimation model and the mental state at a time of generation of the face image; and
generating an integrated estimation result obtained by integrating the predetermined number of the estimation results of the mental state.

9. (canceled)

10. A non-transitory computer readable storage medium storing a program for causing a computer to execute processing comprising:
acquiring a mental state feature amount as a feature amount calculated from a face image of a subject and used for estimation of a mental state of the subject;
acquiring a predetermined number of estimation results of the mental state based on the mental state feature amount and a predetermined number of mental state estimation models each trained using face images in which face directions are different,
wherein each of the predetermined number of the mental state estimation models is a model that has learned a relationship between the mental state feature amount of the face image in which the face direction is associated with the mental state estimation model and the mental state at a time of generation of the face image; and
generating an integrated estimation result obtained by integrating the predetermined number of the estimation results of the mental state.

11. (canceled)

12. The mental state estimation device according to claim 5,
wherein the at least one processor is configured to execute the instructions to determine the weight based on the face direction feature amount and a weight calculation model, and
wherein the weight calculation model
stores Gaussian distributions of representative face direction feature amounts of the predetermined number of the estimation results, and
calculate a confidence interval of the face direction feature amount input to the weight calculation model for each of the Gaussian distributions, and set the weight according to the calculated confidence interval.
